(19) United States
(12) Patent Application Publication
(10) Pub. No.: US 2009/0203785 A1
(43) Pub. Date: Aug. 13, 2009

(54) METHOD AND PROCESS FOR PRODUCING YOUTHFUL-APPEARING, SMALL-PORED, AND SMOOTH SKIN

(76) Inventor: Diana J. Parsons, Berkeley, CA (US)

Correspondence Address: BUSH INTELLECTUAL PROPERTY LAW GROUP, LLC, P.O. BOX 381146, BIRMINGHAM, AL, 35238, US

(21) Appl. No.: 12/421,792

(22) Filed: Apr. 10, 2009

Related U.S. Application Data
(63) Continuation-in-part of application No. 10/735,362, filed on Dec. 12, 2003.
Application No. 10/735,362 is a continuation-in-part of application No. 10/047,335, filed on Jan. 14, 2002, now abandoned.

(51) Int. Cl. A61K 31/203

(52) U.S. Cl. 514/559

(57) ABSTRACT

A method and process for producing youthful appearing, small-pored, and smooth skin by pre-treating the skin topically with retinoic acid, applying carbon particles on the skin, removing the carbon particles by exploding them with laser light, and post-treating the skin topically with retinoic acid. When treating the face, removing the carbon particles requires only about 4 minutes. The laser light itself does not injure the skin. Treatment by explosion of the carbon particles stimulates the production of collagen in the high dermis leaving the epidermis intact, with no removal of stratum corneum. Repeated intermittent treatments of exploding carbon particles and topical application of retinoic acid to the skin produce the desired results in the skin indefinitely as long as the treatment and applications are continued.

### CROSS-REFERENCE TO RELATED APPLICATIONS

This Application is a Continuation-in-Part of application Ser. No. 10/735,362, filed on Dec. 12, 2003, which is a Continuation-in-Part of application Ser. No. 10/047,355, filed on Jan. 14, 2002, now abandoned.

### BACKGROUND OF THE INVENTION

1. Field of the Invention

The present invention relates generally to laser treatments of the skin and, more particularly, to laser-induced explosions of carbon particles on the surface of the skin to produce a chronic wound in the high dermis, leaving the epidermis intact, with topical pretreatment and post-treatment of the skin with retinoic acid.

2. Technical Background

Laser light treatment of the skin is used to rejuvenate the skin, remove pigments and hair, and treat infection. In the field of dermatology and plastic surgery the use of lasers is principally based upon two types of mechanisms: a thermal effect where the laser light energy is converted into heat energy, or a mechanical effect where the laser light energy is converted into shockwaves in the skin.

Most laser treatments use thermal-based laser therapy to vaporize the superficial wrinkles and top layers of the skin so that new collagen and skin can be naturally provided in a healing response. The procedure is performed under either local or general anesthetic, takes a few hours to complete, and recovery takes one to two weeks. During the first week patients may suffer a sensation of intense heat on the skin. Severe burns can occur and result in permanent scarring. Bacterial and yeast infection have been reported and can also lead to scarring. Additional potential complications include changes in pigmentation and herpes infection.

Retinoic Acid (Retin-A, Tretinoin) has also been used as a topical dermal treatment at relatively high concentrations and/or frequency of application to improve the appearance and texture of the skin. Retinoic Acid thins the stratum corneum, increases the thickness of the epidermis, and increases the production of collagen in the dermis. However, at high concentrations and/or frequency of application, Retinoic acid causes skin redness and sensitivity to the sun. Repeated use can cause loss of pigment, painful irritation, dryness, swelling of the skin, and contact dermatitis.

In an effort to overcome these drawbacks of laser therapy and to avoid the unwanted side effects of retinoic acid, Tankovich et al. (U.S. Pat. No. 6,036,684) developed a method of laser-induced explosions of carbon particles on the skin using a Q switched Nd:YAG laser with a wavelength of 1064 nm and an exposure of 2.5 J/cm². Exposure of the skin to this type of laser treatment by itself has no effect on the skin because the skin has no inherent target at the 1064 nm wavelength. However, when an activating solution of graphite or carbon particles (contaminant) suspended in baby oil is applied to the skin, these particles become the target of this 1064 nm wavelength, exploding when exposed to the laser light. The explosion of the carbon particles by the laser light can be adjusted to produce removal of stratum corneum, and/or a localized mechanical injury in the hair follicles and pores of the skin. The laser light itself does not produce significant injury to the skin tissue because the laser light which is not absorbed into the carbon particles is harmlessly dissipated in the skin. The low fluence of the laser light (2.5 J/cm²) leaves the epidermis intact and the typical adverse effects of laser treatment do not occur. Although the laser-induced exploding carbon particle process of Tankovich is considerably safer than the standard laser treatments, this laser-induced exploding particle process is relatively ineffective in treating the skin and has not been commercially successful. To obtain any improvement in the appearance of the skin with the Tankovich method, the stratum corneum needs to be removed (peeled). Beneficial effects on skin of patients are not sustained and the method cannot be used repeatedly because repeated removal of stratum corneum can produce permanent damage to the skin. What is needed to produce long-term improvement in the appearance of the skin is a means for producing adequate new collagen deposition in the high dermis chronically to produce youthful-appearing, small-pored, smooth skin, while leaving the epidermis intact.

### SUMMARY OF THE INVENTION

The present invention provides a process and method for producing skin rejuvenation and therapy by pretreating the skin with retinoic acid; producing a chronic wound in the high dermis using laser-induced explosion of carbon particles on the surface of the skin while leaving the epidermis intact; maintaining skin rejuvenation and therapy chronically by repeating the injury in the high dermis at least once per year; and by topically applying retinoic acid bi-weekly. A Q-switched Nd:YAG laser with a wave length of 1064 nm is used at 2.5 J/cm² to explode the carbon particles on the surface of the skin. A single treatment of the face can be completed in 4 minutes without removing stratum corneum, without anesthesia, with no need for a period of recovery, and at a relatively low cost.

An advantage of the present invention is the production of a chronic wound in the high dermis with no damage to the epidermis and with no removal of stratum corneum.

Another advantage of is the enhancement of laser-induced collagen deposition in the high dermis with retinoic acid.

Another advantage is the chronic life-time rejuvenation and therapy of the skin.

Another advantage is the removal and prevention of acne by suppression of sebaceous glands and reduction of skin pore size.

Another advantage is the use of laser light at a low energy level.

Another advantage is the use of retinoic acid without side effects.

Another advantage is therapeutic removal of infections of the skin.

Another advantage is the chronic rejuvenation and therapy of the skin at a relatively low cost.

Another advantage is that a single treatment of the skin of the face can be completed within four minutes.

### BRIEF DESCRIPTION OF THE DRAWINGS

FIG. 1 describes the method of the present invention.

FIG. 2 illustrates a cross sectional view of the skin and the role of heat in the method of the present invention.

FIG. 3 describes the process of the present invention.

FIG. 4 shows the effects of the laser-induced carbon particle explosion process of the present invention on the restoration of the skin of a patient suffering from acne vulgaris.

### DETAILED DESCRIPTION OF THE INVENTION

While the following description details the preferred embodiments of the present invention, it is to be understood that the invention is not limited in its application to the details of construction and arrangement of the parts illustrated in the accompanying drawings, since the invention is capable of other embodiments and of being practiced in various ways.

Tankovich et al (U.S. Pat. No. 6,036,684) performed skin biopsy studies following their laser-induced carbon particle explosion treatments. These studies confirmed that there was no significant injury to the epidermis underlying the stratum corneum. However, they observed new collagen fiber formation in the upper part of the dermis immediately below the epidermal basal membrane. See column 5, lines 1-13 of Tankovich et al. Tankovich et al. had no explanation for this effect. I have studied this effect and have discovered that it is produced by a selective wounding of the high dermis. The extent of the injury in the high dermis resulting from exploding carbon particles in the epidermis was insufficient to induce adequate collagen deposition during wound healing. Consequently, the Tankovich method does not produce a sustained improvement in the appearance of the skin. In addition, the Tankovich method will produce no improvement in the appearance of the skin unless some of the stratum corneum is removed (peeled).

Wound healing in the skin occurs over a year's time after an initial wounding, regardless of the means by which the wounding is induced. In the first five days the wound is comprised of inflammatory cells and new blood vessels. Then an immature form of collagen is laid down parallel to the skin surface. For six months the body shifts this collagen around, trying to identify the strongest repair. Around six months after the initial wounding, this immature collagen is replaced with mature collagen protein oriented perpendicular to the skin surface. Three months into this process, or months ten, eleven, and twelve from the initial wounding, these protein strands cross-link. This effect is visible on the skin's surface as an apparent shrinkage of the size of the scar to one third its original size.

Twelve months from the initial injury, the wound becomes quiescent. However, any additional insult to the wound site during the first six months of repair causes a signal for a stronger permanent repair. Any additional insult to the wound site during the second six months results in additional mature collagen in the wound. As long as some injury is repeated before twelve months has elapsed, the site becomes a chronic wound producing more collagen, undergoing more cross-linking, and more total surface area shrinkage.

The method and process of the present invention described herein outlines a series of treatments using the Tankovich laser system and activating solution of carbon particles to maximize the initial wounding signal selectively in the high dermis during the first six months of application. In order to produce adequate collagen deposition in the high dermis, beginning at the sixth month from the initial wounding, a series of booster carbon explosion treatments is given to produce additional production of collagen. At a minimum, one booster carbon explosion treatment is given before twelve months has elapsed to keep the wound active. The overall process requires a plurality of wounding events and thus necessitates at least two carbon explosion treatments so that the high dermis may be selectively maintained in a chronically wounded condition, whereby the wound is prevented from healing. If the wound is not kept active, it will be necessary to repeat the initial wounding series of the first six months in order to continue the collagen production process chronically.

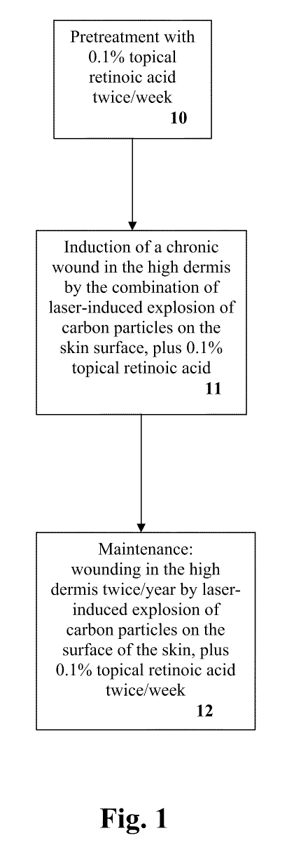

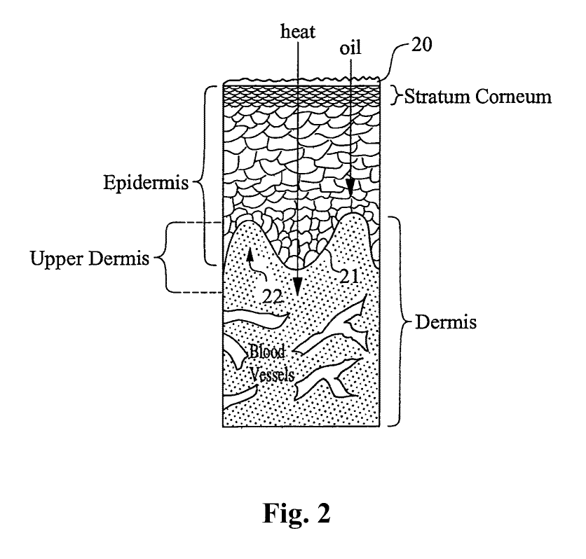

The first step of the method of the present invention is shown in FIG. 1, Step 10, which is a topical pretreatment of the skin with a collagen inducing agent and an angiogenesis inducing agent, preferably, retinoic acid, and in particular its nongeneric form, such as Retin-A (tretinoin). Retinoic acid returns the architecture of the skin back to its more youthful form. As skin ages, the hill and valley nature of the dermalepidermal junction 21 flattens (see FIG. 2). Topical retinoic acid restores the up and down pattern at the junction 21. In youth, the basal cells which originate at the dermalepidermal junction 21 create the layers of the epidermis every three weeks. With aging, this slows to four to six weeks. Topical retinoic acid on the skin returns the process to every three weeks. The topmost layer of epidermal cells, the stratum corneum, is comprised of dead cells held together by cell glue. With age, this layer thickens making the skin less fresh-appearing and making the pores larger. Topical retinoic acid directly attacks the cell glue, thinning the stratum corneum and making the pores smaller by reducing the amount of debris therein. In my studies I have discovered that the relatively low concentrations of 0.05% to 1% retinoic acid in a topical formulation have the properties of inducing new collagen and new blood vessel formation in the high dermis. The pre-treatment period is one to four weeks, preferably two weeks, with retinoic acid being applied twice a week with at least two days between each application. The laser-induced explosion of carbon articles on the skin will not be effective without this pre-treatment application of topical retinoic acid.

The second step 11 in the method of the present invention shown in FIG. 1 is the creation of a chronic wound selectively in the high dermis by the method described in Tankovich et al (U.S. Pat. No. 6,036,684, col 4, lines 5-34) in which carbon particles on the skin are exposed to laser pulses at a frequency of about 1 to 2 Hz. Explosion of the carbon particles with only one or two pulses of laser light will not cause removal of the stratum corneum. The laser light does not interact directly with the skin but instead interacts with the carbon particles on the skin. The carbon particles have the properties of absorbing the laser light and exploding. The carbon or graphite particles are in oil 20 which is applied to the skin (see FIG. 2). Once the carbon particles are applied to the skin, the laser-induced explosion of the carbon particles can begin. The energy from the laser is adjusted to be just sufficient to cause the particles to explode. The heat from the explosion of the carbon particles will induce a wound relatively selectively in the rete peg area of the high dermis 22 initiating a normal wound healing process in the high dermis. The epidermis is left intact with no loss of stratum corneum. The carbon particles on the skin can be exposed to laser pulses at a frequency of about 1 or 2 Hz by making one pass of the laser light across the carbon particles. This process does not remove skin or hair because the carbon particles must be exposed to at least 3 pulses of laser light to remove the stratum corneum, and to 4 or 5 pulses to remove hair. In order to produce a sufficient degree of injury to the high dermis, the laser-induced explosion of carbon particles is produced several times over a six month period, preferably six times over a six month period. During this first six months the retinoic acid is applied topically twice per week as described above.

Due to the use of retinoic acid, the wounding events in the high dermis produce much more collagen than would be produced by such events without the use of retinoic acid. This is so even though the application of retinoic acid by itself is relatively ineffective due to the low concentrations and intermittent applications. Furthermore, the retinoic acid causes the creation of a blood vessel supply to support the collagen. The build up of new collagen in the high dermis 22 in response to the laser-induced explosion of the carbon particles and topical retinoic acid treatment thickens the skin and increases its turgor resulting in smaller skin pores. Broken blood vessels, angiomata, and ice pick scars or suture marks are also minimized, being crowded out by the new collagen. Easy bruisability of the skin is lessened by the collagen build up. Lips become redder, fine lines are minimized, and dark circles around the eyes are minimized. As the new collagen cross-links major wrinkle lines, such as nasal labial folds and perioral and perioccular lines (caused by the underlying attachment of muscles to the deep dermis), marionette lines, sunken-in corners of the mouth, and forehead, temporal, and glabellar lines, appear to melt away into the new skin. The new skin has a glowing quality resulting from the pulsating nature of the wounding process in the high dermis and the mineral oil containing the carbon particles. Retinoic acid and exploding carbon particles work together to minimize bound differential pigmentation caused by sun damage and hormones (mylasma). Redness from inflammation, as in acne vulgaris, acne rosacea, or maturing scars or striae is also reduced.

Because of the closeness of the sebaceous glands to the skin surface and because of the new collagen formation which reduces the size of the skin pores, the method of process of the present invention is useful in the treatment of sebaceous gland disorders such as acne. The secretion from the sebaceous glands is reduced and the smaller skin pore size prevents bacteria from entering the skin pores. In addition, heat generated from the explosion of the carbon particles is sufficient to kill bacteria, fungi, and viruses on the skin's surface or in the high dermis. This also allows treatment of conditions such as, for example, plantar warts, herpes cold sores, athlete's foot, psoriasis, and eczema.

The third step 12 in the method of the present invention, depicted in FIG. 1, is the long term continued chronic maintenance of the high dermis in a wounded condition in order to produce a sustained rejuvenation of the skin and treatment of skin conditions. This is accomplished by application of exploding carbon particles on the surface of the skin at least once a year and preferably twice, along with the continued application of topical retinoic acid to the skin twice per week as described above. The maintaining of the high dermis in a chronically wounded condition causes the high dermis to be in a chronic state of repair, whereby new collagen is produced in the skin above the level of inherent collagen. The repeated creation of new collagen is beneficial since the inherent collagen has usually been damaged by sunlight.

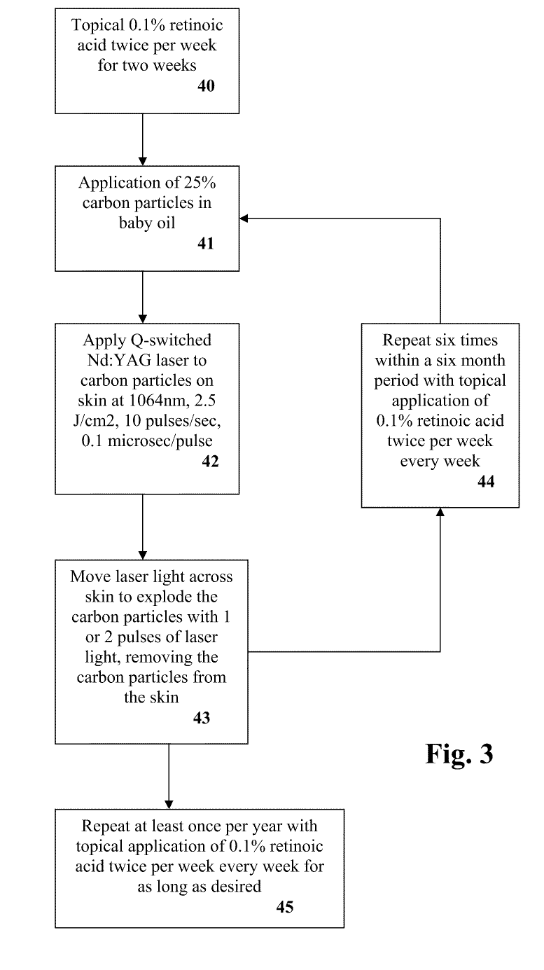

The process of the present invention for treating the skin is shown in FIG. 3. In the first step 40, pre-treatment is initiated with topical retinoic acid applied to the skin at least twice per week. The concentration of retinoic acid in the topical formulation is between about 0.05% to 1% preferably about 0.1%. The pre-treatment period is, preferably, two weeks. In the next step 41, a solution of carbon particles in baby oil is applied to the skin. The carbon particle-oil ratio may range from about 1:1 to 1:9, preferably 1:4, i.e., about 25 percent carbon particles suspended in about 75 percent oil by weight. In the next steps 42 and 43 a laser beam is scanned over the skin treated with the carbon particle solution so as to clean substantially all of the carbon particles from the skin surface by exploding or fracturing the carbon particles. This scanning process takes from about 2 to 10 minutes to complete on the face, usually about 4 minutes, exposing the carbon particles only to 1 or 2 pulses of laser light.

It is preferred to use a Q-switched neodymium:yttrium-aluminum-garnet (Nd:YAG) laser for the process of the present invention. The wave length of the radiation may range from about 800 nM to 1200 nM, preferably about 1064 nM. The frequency of the pulses from the laser range from about 1 to 20 per second, preferably, about 10 per second. The duration of each pulse ranges from about 0.001 to 1 microseconds, preferably about 0.1 microseconds. The fluence or exposure of the skin treated with the carbon particles ranges from about 1 to 3 J/cm².

In the next step 44 the laser-induced explosion of the carbon particles on the surface of the skin is repeated several times to create a chronic wound in the high dermis 22 (see FIG. 2). The process may be repeated 2 to 12 times within a six month period, preferably six times. During this time retinoic acid is applied to the skin 1 to 4 times per week, preferably 2 times per week, with 2 to 3 days between applications. The application of retinoic acid may be varied in an equivalent manner on a monthly basis, i.e., 4 to 16 applications per month, preferably 8 applications per month, where the applications may all be applied within one or two weeks of a given month, but preferably, applied each week of a month.

In the next step 45 the laser-induced explosion of the carbon particles is repeated at least once in the next six months and thereafter at least once per year, but, preferably twice per year. The yearly carbon explosion treatments will maintain a chronic wound in the high dermis. At the same time, topical retinoic acid is applied weekly to the skin as described above. The continued combination of intermittent explosion of carbon particles on the skin and topical retinoic acid application will maintain the rejuvenation and therapeutic results in the skin indefinitely.

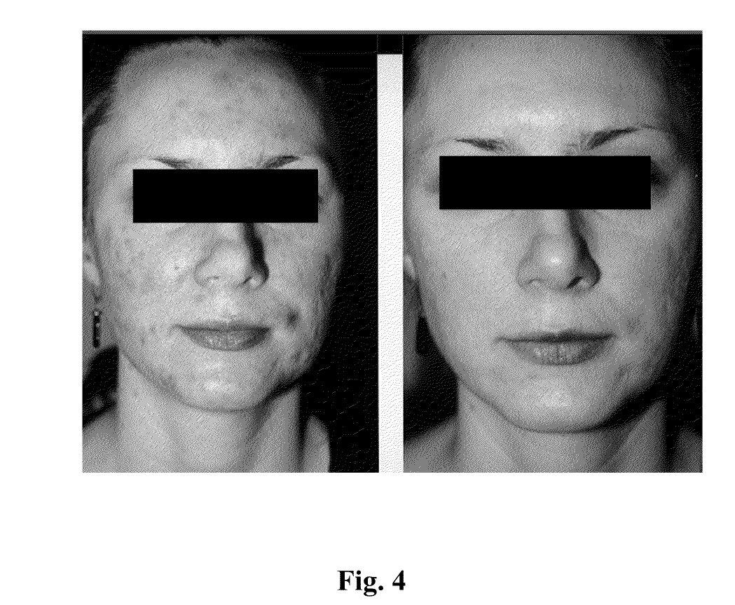

FIG. 4 shows, by way of example, the effects of the combination of topical retinoic acid and exploding carbon particles on the surface of the skin of a patient suffering from acne vulgaris. The before picture (on the left) presents the patient's skin prior to treatment and the after picture (on the right) shows the patient's skin after the first six months of treatment of the skin with the method and process of the present invention described above. The lesions and scars are substantially reduced in severity and the process of acne formation has been completely prevented. Furthermore, this has been accomplished leaving the epidermis intact and normal, with no removal of stratum corneum. The process enables the epidermis to be left intact through the use of the Q-switched laser described above, since the carbon particles on the skin are exposed to only 1 or 2 pulses of laser light. The pulses of heat emitted by the exploding particles on the surface of the skin are deposited in the high dermis. These pulses of heat wound, stimulate, or inflame the high dermis to produce collagen, but do not burn the high dermis. Due to the low energy of the laser light, the laser light does not burn the epidermis or high dermis. Continued chronic treatment with the method and process of the present invention will provide further improvement in the appearance of the skin as long as desired.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. For example, agents other than retinoic acid which induce collagen and blood vessel growth may be used in place of retinoic acid. Any laser system capable of exploding or rupturing a contaminant on the surface skin without injuring the epidermis and without removing stratum corneum can be used in the present invention. The activating solution can be made with any suitable oil and with any suitable contaminant.

It will be understood that various changes in the details, materials, and arrangements of the parts which have been described and illustrated above in order to explain the nature of this invention may be made by those skilled in the art without departing from the principle and scope of the invention as recited in the following claims.

1. A process for producing sustained skin rejuvenation, comprising the steps of:
a) initially applying retinoic acid to the skin;
b) exploding carbon particles on the surface of the skin with laser light, said laser light having a pulse duration of about 0.001 to about 1 microsecond, a wave length of from about 800 nm to 1200 nm, and a fluence of from about 1 to 3 J/cm², and wherein said carbon particles are exploded on the surface of the skin with only 1 or 2 pulses of said laser light, leaving the epidermis intact and undamaged, and producing a wound selectively in the high dermis;
c) applying retinoic acid to the skin 4 to 16 times per month after step b); and
d) repeating step b) at least once every 6 months during step c),
wherein steps c) and d) produce a sustained rejuvenation of the skin.

2. The process of claim 1 wherein the step of initially applying retinoic acid to the skin comprises two weeks.

3. The process of claim 1 wherein the step of exploding carbon particles on the surface of the skin with laser light after initially applying retinoic acid to the skin is performed two to twelve times within a six month period.

4. The process of claim 3 wherein the step of exploding carbon particles on the surface of the skin with laser light after initially applying retinoic acid to the skin is performed six times within a six month period.

5. The process of claim 1 wherein said laser light has a pulse frequency of from about 1 to 20 per second.

6. The process of claim 1 wherein said laser light has a wave length of about 1064 nM, a pulse frequency of about 10 per second, a pulse duration of about 0.01 microsecond, and a fluence of about 2.5 J/cm².

7. The process of claim 1 wherein said retinoic acid is applied topically at a concentration of from about 0.05% to 1% without producing side effects.

8. The process of claim 1 wherein said process is used to improve the appearance of broken blood vessels, angiomata, scars, lines, wrinkles, pigmentation, and redness in the skin.

9. The process of claim 1 wherein said process is used on acne, warts, cold sores, eczema, psoriasis, and infections of the skin.

10. The process of claim 6 wherein said laser light is produced by a Q-switched Nd:YAG laser.

11. The process of claim 1 wherein exploding said carbon particles on the surface of the skin produces a wound in the rete peg area of the high dermis.

12. The process of claim 1 wherein exploding said carbon particles on the surface of the skin does not remove stratum corneum.

13. A process for producing sustained skin rejuvenation, comprising the steps of:
a) initially applying retinoic acid to the skin;
b) exploding carbon particles on the surface of the skin with laser light, said laser light having a pulse duration of about 0.001 to about 1 microsecond, a wave length of from about 800 nm to 1200 nm, and a fluence of from about 1 to 3 J/cm², and wherein said carbon particles are exploded on the surface of the skin with only 1 or 2 pulses with said laser light, leaving the epidermis intact and undamaged, and producing a wound selectively in the high dermis;
c) applying retinoic acid to the skin 4 to 16 times per month after step b); and
d) repeating step b) at least once every 6 months during step c),
wherein steps c) and d) produce a sustained rejuvenation of the skin by maintaining a wound chronically in the high dermis.

14. The process of claim 13 wherein the step of initially applying retinoic acid to the skin comprises two weeks.

15. The process of claim 14 wherein the step of exploding carbon particles on the surface of the skin with laser light after initially applying retinoic acid to the skin is performed two to twelve times within a six month period.

16. The process of claim 15 wherein the step of exploding carbon particles on the surface of the skin with laser light after initially applying retinoic acid to the skin is performed six times within a six month period.

17. The process of claim 16 wherein said laser light has a pulse frequency of from about 1 to 20 per second.

18. The process of claim 17 wherein said laser light has a wave length of about 1064 nM, a pulse frequency of about 10 per second, a pulse duration of about 0.01 microsecond, and a fluence of about 2.5 J/cm².

19. The process of claim 18 wherein said retinoic acid is applied topically at a concentration of from about 0.05% to 1% without producing side effects.

20. The process of claim 19 wherein said process is used to improve the appearance of broken blood vessels, angiomata, scars, lines, wrinkles, pigmentation, and redness in the skin.

21. The process of claim 20 wherein said process is used on acne, warts, cold sores, eczema, psoriasis, and infections of the skin.

22. The process of claim 21 wherein said laser light is produced by a Q-switched Nd:YAG laser.

23. The process of claim 22 wherein exploding said carbon particles on the surface of the skin produces a wound in the rete peg area of the high dermis.

24. The process of claim 23 wherein exploding said carbon particles on the surface of the skin does not remove stratum corneum.
